(19) United States
(12) Patent Application Publication
(10) Pub. No.: US 2012/0109240 A1
(43) Pub. Date: May 3, 2012

(54) AUTOMATIC ADJUSTMENT OF ARRHYTHMIA DETECTION PARAMETERS

(76) Inventors: Xiaohong Zhou, Woodbury, MN (US); Robert Stadler, Shoreview, MN (US); Bruce D. Gunderson, Plymouth, MN (US); Walter H. Olson, North Oaks, MN (US)

(21) Appl. No.: 12/915,877

(22) Filed: Oct. 29, 2010

(51) Int. Cl. A61N 1/39; A61N 1/365

(52) U.S. Cl. 607/7; 607/62; 607/6

(57) ABSTRACT

Methods and/or devices for initiating an automatic adjustment of arrhythmia detection parameters (e.g., upon delivery of cardiac therapy after detection of VT/VF).

The disclosure herein relates to methods for detecting arrhythmias and adjusting one or more arrhythmia detection parameters, and further to devices for performing such processes.

Ventricular tachycardia (VT) and ventricular fibrillation (VF) may be serious, life-threatening forms of cardiac arrhythmias. Implantable cardioverter defibrillators (ICD) are capable of automatically detecting arrhythmias and delivering anti-arrhythmia therapies. Anti-tachycardia pacing therapy (ATP) or defibrillation/cardioversion shock therapy (e.g., high-energy shock therapy) may be used to treat VT and VF. Ventricular tachycardia termination may be referred to as “cardioversion,” and ventricular fibrillation termination may be referred to as “defibrillation.”

Detection of arrhythmias may be determined, for example, by comparing one or more monitored physiological parameters of a patient (e.g., heart-related physiological parameters) to one or more predetermined, programmable detection parameters. For example, the monitored physiological parameters may include intervals between monitored electrical cardiac events in the atria (e.g., P-waves) and/or ventricles (e.g., R-waves). The intervals between two monitored electrical cardiac events (such as R-R intervals or P-P intervals) may be compared to detection parameters (e.g., detection intervals). For example, monitored R-R intervals may be compared to one or more detection intervals, e.g., VT detection interval, a fast VT detection interval, a VF detection interval, etc. A detection interval may be defined as a threshold value, which may be compared to (e.g., less than or greater than) the monitored intervals (e.g., R-R intervals) for various arrhythmia detections.

For example, if the monitored R-R interval is less than one of such detection intervals, it is classified as such for purposes of meeting a specified number of intervals for detection of VT/VF. In other words, VT/VF may be detected when the number of such intervals within a detection interval range exceeds the specified number of intervals. The specified number of intervals may be referred to as the number of intervals to detect VT/VF (NID). For example, VT may be detected if 16 consecutively monitored intervals are less than 400 milliseconds (ms). In this example, the NID is 16 and the detection interval is 400 ms. Further, VF may be detected if 18 of the last 24 monitored intervals are less than 320 ms.

Patients may also experience non-sustained arrhythmias, which terminate spontaneously without any medical intervention. Arrhythmia detection is generally absolute such that an arrhythmia is either detected or not detected. The difference between a sustained arrhythmia requiring treatment and a non-sustained arrhythmia that spontaneously terminates is generally determined by the fixed or pre-selected arrhythmia detection parameters (e.g., programmed by a clinician). For example, if the NID is 16, sustained arrhythmias may be indicated by monitored intervals within a detection interval range that continue longer than 16 intervals. Sustained arrhythmias are generally treated by ATP and/or shock therapy.

### SUMMARY

The disclosure herein relates to methods for detecting an arrhythmia and initiating automatic adjustment of one or more arrhythmia detection parameters in response to delivery of cardiac therapy to treat the detected arrhythmia.

One exemplary implantable medical device disclosed herein for use in delivering therapy to a patient's heart may include sensing apparatus configured to monitor physiological parameters of a patient (e.g., at least one electrode to monitor the electrical activity of the patient's heart), a sensing module coupled to the sensing apparatus and configured to receive the monitored physiological parameters, a therapy delivery module configured to deliver cardiac therapy to the patient, and a control module coupled to the sensing module and to the therapy delivery module. The control module may be configured to provide one or more VT/VF detection parameters usable to detect at least one cardiac condition (e.g., ventricular tachycardia, ventricular fibrillation, etc.) and detect the at least one cardiac condition based on the one or more VT/VF detection parameters using the monitored physiological parameters. The control module may be further configured to initiate an automatic adjustment of at least one of the one or more VT/VF detection parameters to raise a threshold for detection of the at least one cardiac condition in response to delivery of cardiac therapy to treat the at least one cardiac condition. The automatic adjustment results in one or more adjusted VT/VF detection parameters, and in at least one embodiment, follows termination of the at least one cardiac condition.

In one or more embodiments of the exemplary devices and methods disclosed herein, the one or more VT/VF detection parameters may include at least one of a number of intervals to detect VT/VF, a detection interval, and an EGM morphology matching score, and the automatic adjustment of at least one of the one or more VT/VF detection parameters may include at least one of increasing the number of intervals to detect VT/VF, increasing/decreasing the detection interval, and adjusting the EGM morphology matching score to raise the threshold for detection of the at least one cardiac condition.

Further, in one or more embodiments of the exemplary devices disclosed herein, the control module may be further configured to revert to previous one or more VT/VF detection parameters from the one or more adjusted VT/VF detection parameters after expiration of an adjustment time period.

Still further, in one or more embodiments of the exemplary devices disclosed herein, the control module may be further configured to evaluate an effectiveness of the one or more adjusted VT/VF detection parameters used during an adjustment time period (e.g., a selectable time period beginning after the automatic adjustment of the at least one of the one or more VT/VF detection parameters) and revert to previous one or more VT/VF detection parameters from the one or more adjusted VT/VF detection parameters or maintain the one or more adjusted VT/VF detection parameters based upon the evaluation of effectiveness of the one or more adjusted VT/VF detection parameters. Further, in at least one embodiment, the sensing apparatus may further include at least one of a pressure sensor to monitor pressure activity of the patient's heart and a perfusion sensor to monitor tissue perfusion of the patient, and the control module may be further configured to evaluate the effectiveness of the one or more adjusted VT/VF detection parameters by analyzing at least one of the pressure activity of the patient's heart and the tissue perfusion of the patient monitored during the adjustment time period.

One exemplary method disclosed herein for use in delivering therapy to a patient's heart may include monitoring physiological parameters of a patient, providing one or more VT/VF detection parameters usable to detect at least one cardiac condition (e.g., ventricular tachycardia, ventricular fibrillation, etc.), detecting the at least one cardiac condition based on the one or more VT/VF detection parameters using the monitored physiological parameters, and initiating an automatic adjustment of at least one of the one or more VT/VF detection parameters to raise a threshold for detection of the at least one cardiac condition in response to delivery of cardiac therapy to treat the at least one cardiac condition. The automatic adjustment results in one or more adjusted VT/VF detection parameters, and in at least one embodiment, follows termination of the at least one cardiac condition.

In one or more embodiments of the exemplary methods disclosed herein, the exemplary methods may include reverting to previous one or more VT/VF detection parameters from the one or more adjusted VT/VF detection parameters after expiration of an adjustment time period.

Further, in one or more embodiments of the exemplary methods disclosed herein, the exemplary methods may include evaluating an effectiveness of the one or more adjusted VTNF detection parameters used during an adjustment time period (e.g., monitoring at least one of pressure activity of the patient's heart and tissue perfusion of the patient monitored during the adjustment time period.) and reverting to previous one or more VTNF detection parameters from the one or more adjusted VTNF detection parameters or maintaining the one or more adjusted VTNF detection parameters based upon the evaluation of effectiveness of the one or more adjusted VTNF detection parameters.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an exemplary system including an exemplary implantable medical device (IMD).

FIG. 2 is a diagram of the IMD of FIG. 1.

FIG. 3 is a block diagram of the IMD of FIG. 1.

FIG. 4 is a flow chart of an exemplary method for use in delivering therapy to a patient's heart, e.g., using the IMD of FIGS. 1-3.

FIG. 5 is a flow chart of an exemplary method for use in conjunction with the method of FIG. 4.

### DETAILED DESCRIPTION OF EXEMPLARY EMBODIMENTS

In the following detailed description of illustrative embodiments, reference is made to the accompanying figures of the drawing which faun a part hereof, and in which are shown, by way of illustration, specific embodiments which may be practiced. It is to be understood that other embodiments may be utilized and structural changes may be made without departing from (e.g., still falling within) the scope of the disclosure presented hereby.

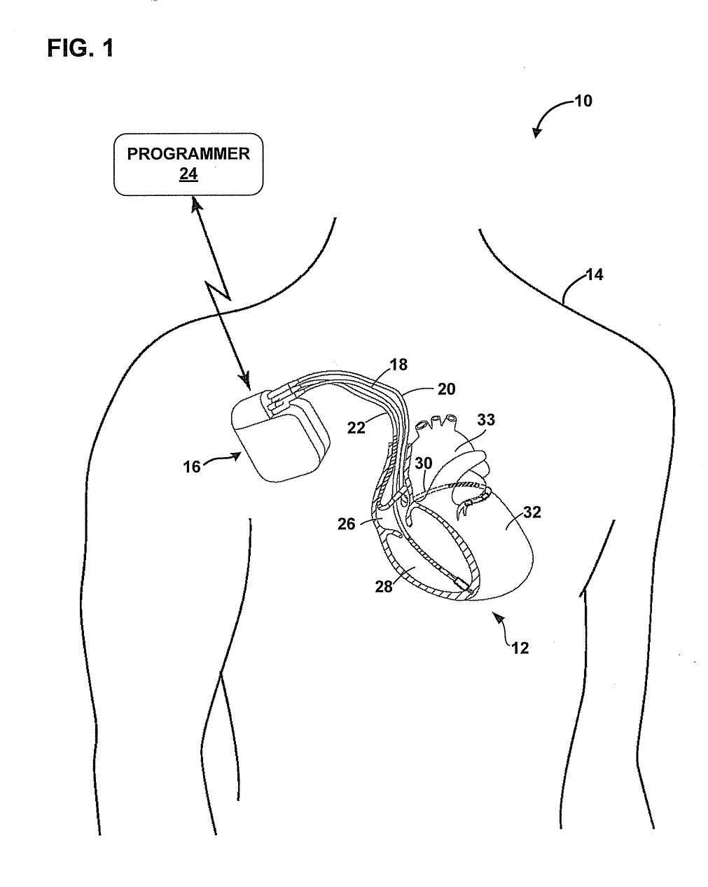

Exemplary methods, devices, and systems shall be described with reference to FIGS. 1-5. It will be apparent to one skilled in the art that elements or processes from one embodiment may be used in combination with elements or processes of the other embodiments, and that the possible embodiments of such methods, devices, and systems using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

FIG. 1 is a conceptual diagram illustrating an exemplary therapy system 10 that may be used to monitor a patient's heart 12 and/or deliver cardiac therapy to the patient 14. Patient 14 may, but not necessarily, be a human. The therapy system 10 may include an implantable medical device 16 (IMD), which may be coupled to leads 18, 20, 22 and a programmer 24. The IMD 16 may be, e.g., an implantable pacemaker, cardioverter, and/or defibrillator, that may provide electrical stimulation to the patient's heart 12 via electrodes coupled to one or more of the leads 18, 20, 22.

The leads 18, 20, 22 extend into the heart 12 of the patient 14 to sense electrical activity of the heart 12 and/or deliver electrical stimulation to the heart 12. In the example shown in FIG. 1, the right ventricular (RV) lead 18 extends through one or more veins (not shown), the superior vena cava (not shown), the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins, the vena cava, and into the right atrium 26 of the heart 12.

The IMD 16 may sense, among other things, electrical signals attendant to the depolarization and repolarization of the heart 12 via electrodes coupled to at least one of the leads 18, 20, 22. In some examples, the IMD 16 provides pacing pulses to the heart 12 based on the electrical signals sensed within the heart 12. The configurations of the electrodes used by the IMD 16 for sensing and pacing may be unipolar or bipolar. The IMD 16 may also provide cardiac resynchronization therapy (CRT), ATP therapy, defibrillation therapy, and/or cardioversion therapy via electrodes located on at least one of the leads 18, 20, 22. For example, the IMD 16 may detect arrhythmia of the heart 12, such as fibrillation of the ventricles 28, 32 and may deliver defibrillation therapy to the heart 12 in the faun of electrical pulses. In some examples, the IMD 16 may be programmed to deliver a progression of therapies, e.g., pulses with increasing energy levels, until a fibrillation of the heart 12 is stopped. Further, the IMD 16 may detect tachycardia and/or fibrillation employing one or more tachycardia and/or fibrillation detection techniques known in the art.

In some examples, the programmer 24 may be a handheld computing device or a computer workstation, which a user, such as a clinician (e.g., a physician, a technician, etc.) and/or patient may use to communicate with the IMD 16. For example, the user may interact with the programmer 24 to transmit data indicative of the effectiveness of the IMD 16 (e.g., effectiveness of one or more VT/VF detection parameters) and to retrieve physiological and/or diagnostic information from the IMD 16.

The IMD 16 and the programmer 24 may communicate via wireless communication using any techniques known in the art. Examples of communication techniques may include, e.g., low frequency or radiofrequency (RF) telemetry, but other techniques are also contemplated.

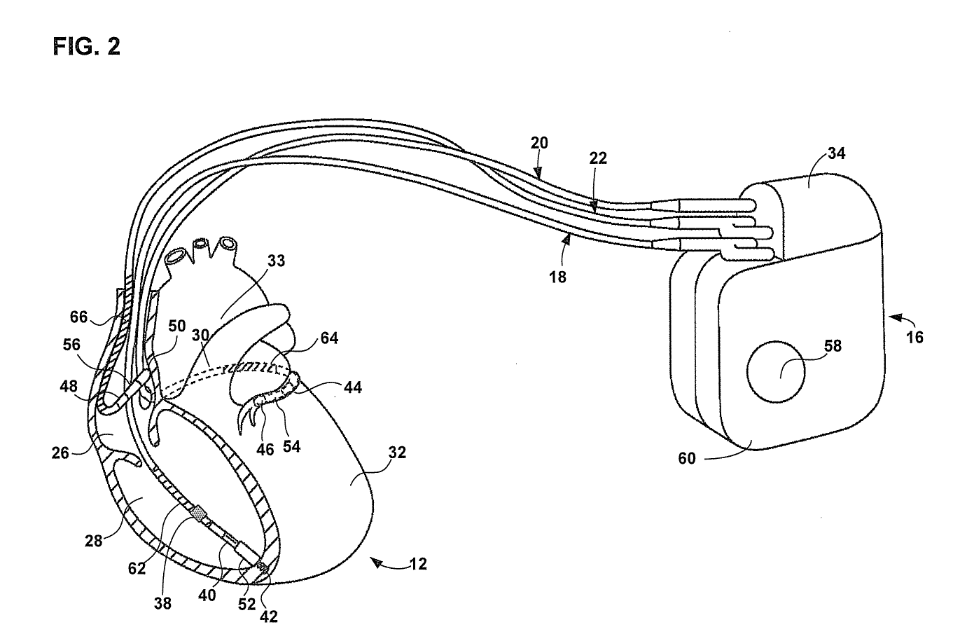

FIG. 2 is a conceptual diagram illustrating the IMD 16 and the leads 18, 20, 22 of the exemplary therapy system 10 of FIG. 1 in more detail. The leads 18, 20, 22 may be electrically coupled to a therapy delivery module, a sensing module, and/or any other modules of the IMD 16 via a connector block 34.

Each of the leads 18, 20, 22 includes an elongated insulative lead body, which may carry a number of concentric coiled conductors separated from one another by tubular insulative sheaths. In the illustrated example, a pressure sensor 38 and bipolar electrodes 40, 42 are located proximate to a distal end of the lead 18. In addition, bipolar electrodes 44, 46 are located proximate to a distal end of the lead 20 and bipolar electrodes 48, 50 are located proximate to a distal end of the lead 22. As shown in FIG. 2, the pressure sensor 38 is disposed in the right ventricle 28 of the patient's heart 12. The pressure sensor 38 may respond to an absolute pressure inside the right ventricle 28, and may be, e.g., a capacitive and/or piezoelectric pressure sensor. In other examples, the pressure sensor 38 may be positioned within other regions of the heart 12 (e.g., the left ventricle) and may monitor pressure within one or more regions of the heart 12, or may be positioned elsewhere within or proximate to the cardiovascular system of the patient 14 to monitor one or more cardiovascular pressures of one or more portions of the patient's heart 12, e.g., associated with mechanical contraction of the heart.

The electrodes 40, 44, 48 may take the form of ring electrodes, and the electrodes 42, 46, 50 may take the faun of extendable helix tip electrodes mounted retractably within the insulative electrode heads 52, 54, 56, respectively. In some examples, e.g., as illustrated in FIG. 2, the IMD 16 may include one or more housing electrodes, such as housing electrode 58, which may be formed integrally with an outer surface of a housing 60 (e.g., hermetically-sealed housing) of the IMD 16 or otherwise coupled to the housing 60.

The leads 18, 20, 22 may also include elongated electrodes 62, 64, 66, respectively, which may take the faun of a coil. The IMD 16 may deliver defibrillation shocks and/or cardioversion pulses to the heart 12 via any combination of the elongated electrodes 62, 64, 66, and the housing electrode 58.

The configuration of the exemplary therapy system 10 illustrated in FIGS. 1-2 is merely one example. In other examples, an exemplary therapy system may include epicardial leads and/or patch electrodes instead of or in addition to the transvenous leads 18, 20, 22 illustrated in FIGS. 1-2. Further, in one or more embodiments, the IMD 16 need not be implanted within the patient 14. For example, the IMD 16 may monitor electrical signals of one or more portions of the heart 12, deliver cardioversion/defibrillation shocks, deliver ATP, and/or perform other therapies to the heart 12 via percutaneous leads that extend through the skin of the patient 14 to a variety of positions within or outside of the patient's heart 12.

In other exemplary therapy systems that provide electrical stimulation therapy to the heart 12, the therapy systems may include any suitable number of leads coupled to the IMD 16, and each of the leads may extend to any location within or proximate to the patient's heart 12. For example, other exemplary therapy systems may include three transvenous leads located as illustrated in FIGS. 1-2, and an additional lead located within or proximate to the left atrium 33. Still further, other exemplary therapy systems may include a lead that extends from the IMD 16 into the right atrium 26 or the right ventricle 28, or two leads that extend into a respective one of the right ventricle 26 and the right atrium 28.

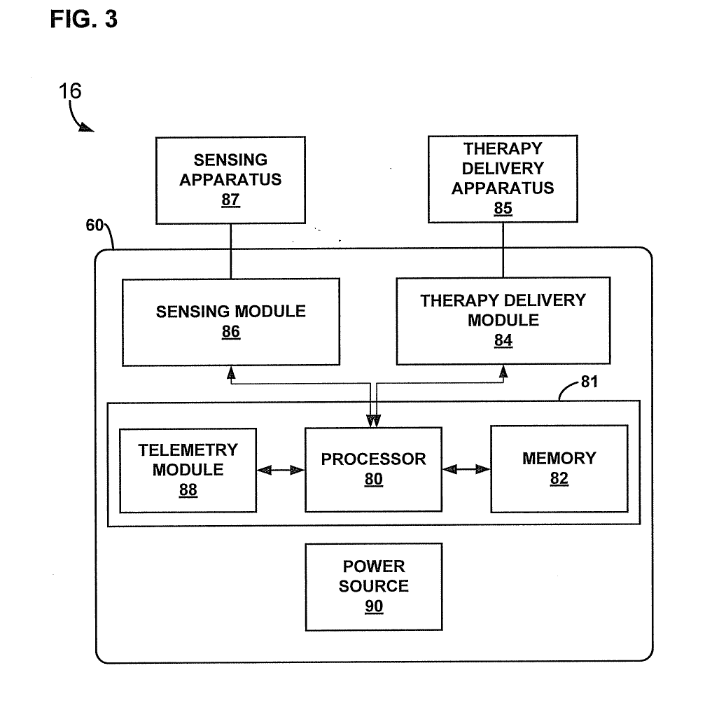

FIG. 3 is a functional block diagram of one exemplary configuration of the IMD 16. As shown, the IMD 16 may include a control module 81, a therapy delivery module 84 (e.g., including a stimulation generator), a sensing module 86, and a power source 90.

The control module 81 may include a processor 80, memory 82, and a telemetry module 88. The memory 82 may include computer-readable instructions that, when executed, e.g., by the processor 80, cause the IMD 16 and the control module 81 to perform various functions attributed to the IMD 16 and the control module 81 described herein. Further, the memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

The processor 80 of the control module 81 may include any one or more of a microprocessor, a controller, a digital signal processor (DSP), an application specific integrated circuit (ASIC), a field-programmable gate array (FPGA), or equivalent discrete or integrated logic circuitry. In some examples, the processor 80 may include multiple components, such as any combination of one or more microprocessors, one or more controllers, one or more DSPs, one or more ASICs, or one or more FPGAs, as well as other discrete or integrated logic circuitry. The functions attributed to the processor 80 herein may be embodied as software, firmware, hardware, or any combination thereof.

The processor 80, or any other portion, of the control module 81 may employ digital signal analysis techniques to characterize the digitized signals stored in memory 82 to recognize and classify the patient's heart rhythm employing any of the numerous signal processing methodologies known in the art. An exemplary tachyarrhythmia recognition mechanism is described in U.S. Pat. No. 5,545,186 issued to Olson et al, which is incorporated herein by reference in its entirety.

The control module 81 is coupled to and controls the therapy delivery module 84, which is configured to deliver therapy (e.g., electrical stimulation therapy) to the patient's heart 12 according to one or more of therapy programs that may be stored in the memory 82. Specifically, the processor 80 of the control module 81 may control the therapy delivery module 84 to deliver electrical pulses with delays, timings, amplitudes, pulse widths, frequency, and/or electrode polarities specified by the one or more therapy programs (e.g., CRT programs, ATP programs, defibrillation/cardioversion programs, etc.).

The therapy delivery module 84 is coupled (e.g., electrically coupled) to therapy delivery apparatus 85 such that the therapy deliver module 84 may use the therapy delivery apparatus 85 to deliver therapy to the patient 14. The therapy deliver apparatus 85 may include, among other therapy delivery devices, the electrodes 40, 42, 44, 46, 48, 50, 58, 62, 64, 66 of the exemplary system of FIGS. 1-2 (e.g., via conductors of the respective leads 18, 20, 22). The therapy delivery module 84 may be configured to generate and deliver electrical stimulation therapy to the heart 12. For example, the therapy delivery module 84 may deliver pacing pulses (e.g., for use in providing ATP and/or CRT) via the ring electrodes 40, 44, 48 coupled to the leads 18, 20, 22, respectively, and/or the helical electrodes 42, 46, 50 of the leads 18, 20, 22, respectively. Further, for example, the therapy deliver module 84 may deliver defibrillation/cardioversion shocks to the heart 12 via at least two of the plurality of electrodes, e.g., electrodes 58, 62, 64, 66. In some examples, the therapy delivery module 84 may deliver pacing, cardioversion, and/or defibrillation stimulation in the form of electrical pulses.

The control module 81 is coupled to and controls the sensing module 86 to receive one or more signals from sensing apparatus 87. The sensing module 86 is coupled (e.g., electrically coupled) to sensing apparatus 87, e.g., to monitor signals from the sensing apparatus 87. The sensing apparatus 87 may include the electrodes 40, 42, 44, 46, 48, 50, 58, 62, 64, 66 to monitor electrical activity of the heart 12, e.g., to provide electrocardiogram (ECG) signals, etc. The sensing apparatus 87 may further include one or more pressure sensors (e.g., the pressure sensor 38), posture sensors (e.g., accelerometers), heart sound sensors, perfusion sensors (e.g., optical perfusion sensors as described in U.S. Pat. App. Pub. No. 2009/0326356 A1 to Kracker, which is incorporated by reference herein in its entirety), etc.

Although not depicted, the sensing module 86 may further include an atrial sense amplifier and a ventricular sense amplifier, which may take the form of automatic gain controlled amplifiers with adjustable sensing thresholds. Exemplary sense amplifiers are disclosed in U.S. Pat. No. 5,117,824 to Keimel et al., which is incorporated herein by reference in its entirety. In at least one embodiment, whenever a signal is received by the atrial sense amplifier that exceeds an atrial sensing threshold, a signal representative of an atrial electrical event is generated and transmitted to the control module 81, and likewise, whenever a signal is received by the ventricular sense amplifier that exceeds a ventricular sensing threshold, a signal representative of a ventricular electrical event is generated and transmitted to the control module.

As described herein, the IMD 16 may be configured to generate and deliver electrical stimulation (e.g., pacing pulses for use in ATP) to the patient's heart 12, and as such, the control module 81 may include a pacer timing and control module, which may be embodied as hardware, firmware, software, or any combination thereof. The pacer timing and control module may include a dedicated hardware circuit, such as an ASIC, separate from the other components, such as a microprocessor, or an executable software module. The pacer timing and control module may include programmable digital counters which control the basic time intervals associated with various single, dual or multi-chamber pacing modes or anti-tachycardia pacing therapies delivered in the atria or ventricles.

For example, the pacer timing and control module may include programmable counters which control the basic time intervals associated with DDD, VVI, DVI, VDD, AAI, DDI, DDDR, VVIR, DVIR, VDDR, AMR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

Intervals defined by the pacer timing and control module may include the AV delay, the VV delay, etc. The AV delay may be defined as the time interval between pacing the atria and pacing the ventricles of the patient's heart 12 and the VV delay may be defined as the time interval between pacing the left ventricle and the pacing the right ventricle of the patient's heart 12. The durations of these intervals may be determined by the processor 80 of the control module 81 in response to stored values in the memory 82 (e.g., nominal AV and/or VV delays, clinician selected AV and/or VV delays, automatically-adjusted AV and/or VV delays, etc.).

The therapy delivery module 84 may include pacer output circuits that are selectively coupled (e.g., using switching circuitry) to any one or more of the electrodes 40, 42, 44, 46, 48, 50, 58, 62, 66 appropriate for delivery of a bipolar or unipolar pacing pulse to one of the portions of the patient's heart 12.

In at least one embodiment, ATP therapy may be delivered to the patient in response to the detection of an arrhythmia by loading a therapy regimen from the memory 82 using the processor 80 into a pacer timing and control module according to the type of arrhythmia detected. In the event that higher voltage cardioversion/defibrillation pulses are required, the control module 81 may activate various cardioversion/defibrillation control circuitry of the IMD 16 to initiate charging of one or more high voltage capacitors. When the voltage of the capacitors reaches a predetermined value, charging may be terminated and a defibrillation and/or cardioversion pulse may be delivered to the patient's heart 12 using one or more selected electrodes depending on the type of cardioversion/defibrillation pulse and pulse wave shape.

The telemetry module 88 of the control module 81 may include any suitable hardware, firmware, software, or any combination thereof for communicating with another device, such as the programmer 24 (FIG. 1). For example, under the control of the processor 80, the telemetry module 88 may receive downlink telemetry data from and send uplink telemetry data to the programmer 24 with the aid of an antenna, which may be internal and/or external. The processor 80 may provide the data to be uplinked to the programmer 24 and the control signals for the telemetry circuit within the telemetry module 88. Further, the telemetry module 88 may provide received data to the processor 80 via a multiplexer.

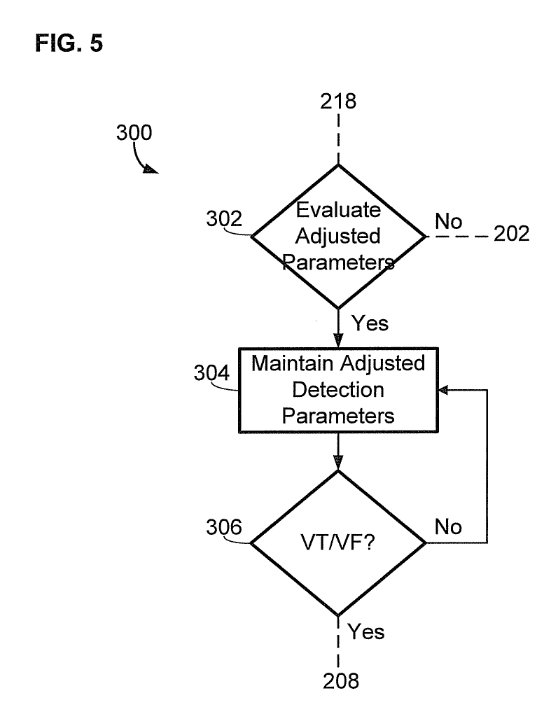

In at least one embodiment, the control module 81 may receive data from or transmit data to an external device, such as the programmer 24, using the telemetry module 88. The data transmitted, e.g., by a clinician or patient, to or from the control module 81 may be indicative of the effectiveness of presently-used arrhythmia detection parameters. For example, if a patient has been negatively affected (e.g., dizziness, syncope, fast palpitation, breathlessness, faint, etc.) after VT/VF detection parameters have been adjusted, data indicating that the adjusted VT/VF detection parameters have been ineffective and/or unsuccessful may be transmitted. Likewise, if a patient has been positively affected or unaffected (e.g., less unnecessary therapy, no ill effects, etc.) after VT/VF detection parameters have been adjusted, data indicating that the adjusted VT/VF detection parameters have been effective and/or successful may be transmitted. Methods using transmission of data indicative of effectiveness of detection parameters is described further herein with reference to method 300 of FIG. 5.

In at least another embodiment, the control module 81 may transmit data from the telemetry module 88 to an external device, such as the programmer 24. The data transmitted may include various monitored physiological parameters (e.g., pressure, perfusion, R-R intervals, P-P intervals, heart rate, number of detected arrhythmias), arrhythmia detection parameters (e.g., detection interval, NID, etc.), and/or therapy parameters (e.g., energy levels, timings, etc.). For example, a clinician may use transmitted data to analyze the patient's condition and/or adjust various parameters (e.g., arrhythmia detection parameters, therapy parameters, etc.) within the IMD 16.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily, weekly, and/or other periodic basis.

Although not depicted, in at least one embodiment, the IMD 16 may include a patient notification system used to notify the patient of various cardiac events. For example, the notification system may notify that patient that an imminent, sustained arrhythmia is predicted. Any known patient notification method may be used such as generating a perceivable somatosensory or twitch stimulation and/or an audible sound. A patient notification system may include an audio transducer that emits audible sounds including voiced statements or musical tones stored in memory and correlated to a programming or interrogation operating algorithm such as generally described in U.S. Pat. No. 6,067,473 issued to Greeninger et al., which incorporated herein by reference in its entirety.

Patients having a sustained VT or VF (VT/VF) may be more likely to experience non-sustained VT/VF following the sustained VT/VF. As such, patients that experience frequent non-sustained VT/VF following sustained VT/VF may be exposed to repeated ATP and/or shock therapy that may be unnecessary because the non-sustained VT/VFs may be identified as sustained VT/VF thereby triggering the ATP and/or shock therapy. Further, such ATP and/or shock therapy can be painful to the patient, consume ICD battery energy, and, in some cases, accelerate or otherwise worsen the severity of a heart condition. Such non-sustained arrhythmias may not require additional therapy or the same type of therapy that a sustained arrhythmia may require. As such, the arrhythmia detection parameters, which detected sustained arrhythmias, may be adjusted such that non-sustained arrhythmias are not detected as sustained arrhythmias.

For example, the methods and/or devices described herein may detect at least one cardiac condition based on one or more arrhythmia detection parameters and initiate an automatic adjustment of at least one of the arrhythmia detection parameters in response to detection of the at least one cardiac condition or delivery of cardiac therapy to treat the at least one cardiac condition.

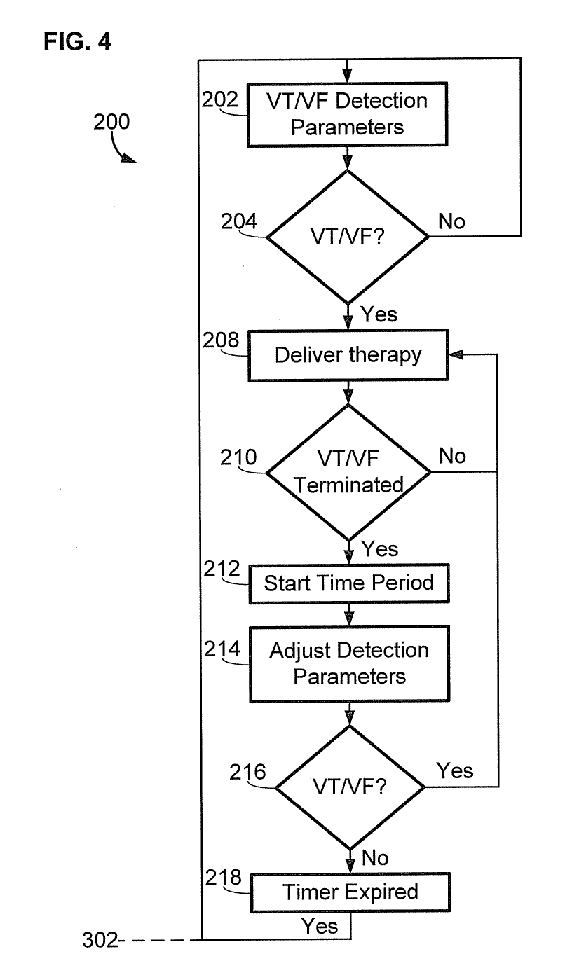

An exemplary generalized method 200 for use in delivering cardiac therapy to a patient's heart using such automatic adjustment is diagrammatically depicted in FIG. 4.

Method 200 is intended to illustrate the general functional operation of the devices and/or systems described herein, and should not be construed as reflective of a specific faun of software or hardware necessary to practice all of the methods described herein. It is believed that the particular form of software will be determined primarily by the particular system architecture employed in the device (e.g., the IMD 16) and by the particular detection and therapy delivery methodologies employed by the device and/or system. Providing software and/or hardware to accomplish the described methods in the context of any modem IMD, given the disclosure herein, is within the abilities of one of skill in the art.

Although not depicted, the method 200 may include a data collection process that is executed concurrently, sequentially, and/or periodically with one or more processes of method 200 described herein. The data collection process may include monitoring electrical activity of one or more portions of a patient's heart using one or more electrodes located within or proximate various locations of the patient's heart (e.g., as described herein with reference to FIGS. 1-2). The monitored electrical activity may be used by method 200 to determine or detect various one or more cardiac conditions of a patient (e.g., VT/VF).

The method 200 includes providing one or more VT/VF detection parameters (block 202). The VT/VF detection parameters may be usable to detect at least one cardiac condition (e.g., VT, VF, etc.). The VT/VF detection parameters may be nominal values. As used herein, values described as “nominal” may be default values that are preset within the IMD 16 or set by a clinician. In other words, nominal values may be initial or starting values that, e.g., may be adjusted in the future.

A VT and/or VF may be detected (block 204) using one or more monitored physiological parameters of a patient's heart and the provided VT/VF detection parameters (block 202). If a VT/VF is detected (block 204), the method 200 may proceed to delivering cardiac therapy to treat the VT/VF (block 208). The cardiac therapy delivered to treat the VT/VF (block 208) may include one or more of ATP, cardioversion shocks, defibrillation shocks, nerve stimulation (e.g., electrical stimulation of the vagus nerve), CRT, drug perfusion, contractility modulation, blood pressure modulation, etc. The method 200 may further continue delivering cardiac therapy to the patient (block 208) until the VT/VF is terminated (block 210).

As described herein, after a patient has undergone a sustained VT/VF episode, it is likely that the patient may undergo one or more non-sustained VTNFs (e.g., VTNFs that terminate by themselves) for a time period after the sustained VT/VF. As such, it may be beneficial to automatically raise the threshold for detection of VT/VF to avoid unnecessary treatment following a sustained VT/VF. In other words, the method 200 may automatically raise the threshold for detection of VT/VF to avoid false positive indications of sustained VT/VF when a non-sustained VT/VF has been detected and in response to therapy being delivered.

Although it has been described herein that the threshold for detection of VT/VF is automatically “raised,” it is to be understood that “raising” the threshold includes automatically modifying (e.g., increasing, decreasing, etc.) one or more VT/VF detection parameters such that detection of VT/VF is less likely after the modification. In other words, after the threshold for detection of VT/VF is automatically raised, VT/VF detection may be more difficult, may require more confirmation, and/or may take a longer period of time than prior to adjustment of the VT/VF detection parameters.

Further, it is to be understood that the disclosure herein may also include automatically lowering (as opposed to raising) the threshold for detection of VT/VF which may include automatically modifying (e.g., increasing, decreasing, etc.) one or more VT/VF detection parameters such that detection of VT/VF is more likely after the modification. In other words, after the threshold for detection of VT/VF is automatically lowered, VT/VF detection may be easier, may require less confirmation, and/or may take a shorter period of time than prior to adjustment of the VT/VF detection parameters. Generally, to lower the threshold for detection of VT/VF, the one or more VT/VF detection parameters must be modified in an opposite fashion than they would be to raise the threshold for detection of VT/VF. Both concepts, raising the threshold and/or lowering the threshold for detection of VT/VF, may be, in other words, modifying the threshold for detection of VT/VF.

To raise the threshold for detection of VT/VF, the method 200 may start an adjustment time period (block 212) and initiate an automatic adjustment of the VT/VF detection parameters (block 214) after the VT/VF has been detected (block 204), therapy has been delivered (block 208), and/or the VT/VF has been terminated (block 210). At least one of the VT/VF detection parameters may be automatically adjusted to raise the threshold for detection of VT/VF. For example, one or more of the following VT/VF detection parameters may be automatically adjusted: NID, detection interval/zone, morphology matching score (e.g., as described in U.S. Pat. No. 6,393,316 to Gillberg et al., which is incorporated herein by reference in its entirety), timing of therapy, hemodynamic thresholds, heart sounds, blood pressure, tissue perfusion, motion thresholds (e.g., using an accelerometer), cognitive thresholds, etc.

As described, adjustments to at least one of the arrhythmia detection parameters (e.g., VT/VF detection parameters) are described as being “automatic.” As used herein, an “automatic adjustment” is an adjustment that occurs in direct response to a triggering event. In other words, an automatic adjustment occurs resulting in an adjustment of one or more VT/VF detection parameters such that the at least one parameter changes in at least some way. For example, if the detection parameter that is to be automatically adjusted is NID and the NID is presently 16, after an automatic adjustment, the NID cannot be 16 and must be another value other than 16. In other words, an “automatic adjustment” of a parameter never results in that particular parameter remaining the same as it was before the automatic adjustment.

In at least one embodiment, the adjustment of the VT/VF detection parameters (block 214) may automatically occur after a selected period of time or heart beats after the delivery of cardiac therapy (block 208) (and/or any other process of method 200). For example, the adjustment of the VT/VF detection parameters (block 214) may occur 30 seconds or 30 heart beats after the delivery of cardiac therapy (block 208).

In at least another embodiment, the number of intervals to detect VT/VF (NID) may be automatically increased in response to delivery of cardiac therapy to treat the detected VT/VF to raise the threshold for detection of VT/VF. For example, the NID may be 16 prior to adjustment. After the delivery of cardiac therapy (block 208) (e.g., following the termination of the VT/VF (block 210)), the NID may be automatically adjusted to 24 (block 214) to, e.g., raise the threshold for detection of VT/VF.

In at least another embodiment, a selected number of intervals out of a number of previous intervals to detect VT/VF may be automatically increased in response to delivery of cardiac therapy to treat the detected VT/VF to raise the threshold for detection of VT/VF. For example, VT/VF may be detected if 18 out of the previous 24 intervals are less than 320 ms (e.g., the detection interval). After the delivery of cardiac therapy (block 208) (e.g., following the termination of the VT/VF (block 210)), the selected number of intervals out of the number of previous intervals to detect VT/VF may be automatically adjusted to 20 (block 214) to, e.g., raise the threshold for detection of VT/VF. Further, the selected number of intervals out of the number of previous intervals may be expressed as a percentage (e.g., 18 intervals out the previous 24 intervals is 75%) and the percentage may be increased (e.g., to 85%) to, e.g., raise the threshold for detection of VT/VF.

In at least another embodiment, the detection interval usable to detect VT/VF may be automatically decreased in response to delivery of cardiac therapy to treat the detected VT/VF to raise the threshold for detection of VT/VF. For example, the detection interval may be 400 milliseconds (ms) prior to adjustment. After at least one of the delivery of cardiac therapy (block 208) and/or the termination of the VT/VF (block 210), the detection interval may be automatically adjusted to 350 ms (block 214) to, e.g., raise the threshold for detection of VT/VF.

At least in some embodiments, the detection interval usable to detect VT/VF may be automatically increased in response to delivery of cardiac therapy to treat the detected VT/VF to lower the threshold for detection of VT/VF. For example, the detection interval may be 320 ms prior to adjustment. After at least one of the delivery of cardiac therapy (block 208) and/or the termination of the VT/VF (block 210), the detection interval may be automatically adjusted to 360 ms (block 214) to, e.g., lower the threshold for detection of VT/VF.

In at least another embodiment, an EGM morphology matching score usable to detect VT/VF may be automatically adjusted in response to delivery of cardiac therapy to treat the detected VT/VF to raise the threshold for detection of VT/VF. For example, an EGM morphology matching score used to detect VT/VF may be 70% (e.g., on a sliding scale of 0% to 10%, with 100% being indicative a proper cardiac function and/or normal/benign sinus rhythm and 0% being indicative of poor cardiac function and/or abnormal/poor sinus rhythm) prior to adjustment. After at least one of the delivery of cardiac therapy (block 208) and/or the termination of the VT/VF (block 210), the EGM morphology matching score may be automatically adjusted (e.g., decreased) to 60% (block 214) to, e.g., raise the threshold for detection of VT/VF. Further, a selected number of intervals having a matching score less than the matching score used to detect VT/VF out of a number of previous intervals may be required to detect VT/VF (e.g., 5 intervals out of the previous 8 intervals). The selected number of intervals having a matching score less than the matching score used to detect VT/VF out of the number of previous intervals may be increased (e.g., to 6 intervals out of the previous 8 intervals) to, e.g., raise the threshold for detection of VT/VF. Still further, the selected number of intervals having a matching score less than the matching score used to detect VT/VF out of the number of previous intervals may be expressed as a percentage (e.g., 6 intervals out the previous 8 intervals is 75%) and the percentage may be increased (e.g., to 85%) to, e.g., raise the threshold for detection of VT/VF.

Further, the values by which the one or more VT/VF detection parameters are automatically adjusted may be based on one or more monitored characteristics of the previous detected VT/VF. In at least one embodiment, the NID may be adjusted based on the number of intervals that occurred during the detected VT/VF. For example, if the detected VT/VF occurred for 24 intervals, the NID may be adjusted to be 120% of the number of intervals of the detected VT/VF, e.g., 29 (e.g., rounded up). In at least another embodiment, the NID may be adjusted based on the duration of the detected VT/VF. For example, if the detected VT/VF occurred for 8 seconds, the NID may be adjusted to the duration of the detected VT/VF divided by the current detection interval multiplied by 120% (e.g., 8 seconds/350 ms×120%=about 27). In other words, one or more of the VT/VF detection parameters may be automatically adjusted by a dynamic value based on one or more characteristics of the previously detected VT/VF.

The adjustment time period started (block 212) following at least one of the detection of VT/VF (block 204), the delivery of cardiac therapy (block 208), and/or the termination of the VT/VF (block 210) may be representative of the amount of time after a sustained VT/VF that a patient is likely to undergo non-sustained VT/VFs. The adjustment time period may be selectable, e.g., by a clinician, such that the adjustment time period may be customized for each individual patient and/or different cardiac conditions. In at least one embodiment, the adjustment time period may be based on previously-monitored heart-related parameters of a patient (e.g., previously-monitored VT/VF clusters).

The adjustment time period may be about a ½ hour to about 24 hours. (e.g., about a ½ hour, about 1 hour, about 2 hours, about 4 hours, about 6 hours, about 12 hours, about 24 hours, etc.) In at least one embodiment, the adjustment time period may be maintained until the patient's next visit to a clinician's office for a checkup or the next remote monitoring session (e.g., using Medtronic CareLink®). Further, the adjustment time period may be adjustable by the method 200, e.g., depending on the efficacy of a present or previous adjustment time period.

Although the processes 208, 212, 214, are depicted as sequential, such processes may be executed in any order including substantially concurrently. For example, the start of the adjustment time period (block 212), and/or the initiation of the automatic adjustment of the VT/VF detection parameters (block 214) may occur concurrently after the VT/VF detection (block 204) or after the VT/VF termination (block 210). Further, for example, the delivery of cardiac therapy (block 208), the start of the timer (block 212), and/or the initiation of the automatic adjustment of the VT/VF detection parameters (block 214) may occur concurrently after the VT/VF detection (block 204).

Although method 200 describes detecting, treating, and/or adjusting detection parameters for VT/VF, the method 200 may detect, treat, and/or adjust parameters for any one or more cardiac condition (e.g., atrial fibrillation, atrial tachycardia, poor contractility, low blood pressure, low tissue perfusion, congestive heart failure, low heart rate, ischemia, etc.).

The method 200 may further automatically adjust the arrhythmia detection parameters after they have already been adjusted if a VT/VF is detected (block 216) prior to the expiration of the adjustment time period (block 218). For instance, if a VT/VF is detected (block 216) after the VT/VF detection parameters have been automatically adjusted (block 214) prior to the expiration of the adjustment time period (block 218), the method 200 may return to delivering cardiac therapy (block 208), starting (e.g., resetting and restarting) an adjustment time period (block 212), and/or automatically adjusting the VT/VF detection parameters (block 214). In other words, the VT/VF detection parameters may be further automatically adjusted to raise the threshold for detection of VT/VF higher than the threshold had previously been raised if VT/VF is detected prior to the expiration of the adjustment time period (block 218). For example, if the NID had already been increased from 16 to 20, the NID may be increased from 20 to 30.

Each of the values that each of the one or more VT/VF detection parameters may be adjusted by may be pre-selected (e.g., by a clinician, preset in the IMD, etc.) and/or may be determined based on various criteria. For example, the method 200 may taken into consideration the number of VT/VFs that have been detected within the adjustment time period, the morphology of EGM signals, heart sounds, blood pressure, cardiac contractility, tissue perfusion, etc.

In at least one embodiment, the NID may be automatically increased (block 214) by a first value, e.g., 4. If VT/VF is again detected (block 216) within the adjustment time period, the NID may be automatically adjusted (block 214) for a second time by a second value, e.g., 2, 4, 6, 8, 10, etc., that is the same or different than the first. In other words, although the threshold for detection of VT/VF may be raised, it may be raised more quickly, less quickly, or the same, after the first automatic adjustment.

Further, if one or more VT/VF detection parameters are adjusted to raise the threshold for detection of VT/VF, it may take a longer period of time to detect sustained VT/VFs. In other words, detection of a sustained VT/VF may be delayed if one or more of the VT/VF detection parameters are adjusted to raise the threshold for detection of VT/VF. As a result, the cardiac therapy delivered to treat the sustained VT/VFs may be adjusted (e.g., increased) to compensate for the delayed detection. For example, the method 200 may include adjusting at least one parameter of cardiac therapy to treat the VT/VF based on the automatic adjustment of at least one of the VT/VF detection parameters (block 214). Exemplary adjustable parameters of cardiac therapy may be energy level of defibrillation therapy, frequency of ATP runs, shock therapy charge time, drug perfusion speed, intensity of nerve stimulation or neuromodulation, etc. In at least one embodiment, the energy level of the defibrillation shock therapy may be increased after the VT/VF detection parameters have been automatically adjusted. In at least another embodiment, the shock therapy charge time may be decreased after the VT/VF detection parameters have been automatically adjusted. In at least another embodiment, the frequency of ATP runs may be increased or the number of ATP sequences may be decreased (e.g., to proceed to shock therapy more quickly) after the VT/VF detection parameters have been automatically adjusted.

After the timer has expired (block 218), the method 200 may return to providing or setting the VT/VF detection parameters (block 202) back to nominal values (e.g., original values, pre-adjustment values, etc.) and/or proceed to an additional learning or evaluation method 300 described herein with reference to FIG. 5.

In at least one embodiment, VT/VF detection may have a fixed, or short, duration to “detect” VT/VF but also have a duration of continuous detection before therapy is delivered (e.g., the duration of continuous detection may be a selectable time period after VT/VF detection but before, e.g., confirmation of VT/VF, delivery of therapy, etc.). In other words, VT/VF detection may be “fast” but may not be immediately followed by therapy. In such embodiments, the selectable duration of continuous detection prior to therapy may be increased such that more continuous detection may occur prior to delivery of therapy to raise the threshold for detection of VT/VF.

The method 300 provides a learning or evaluation process to determine whether to reset the VT/VF detection parameters back to nominal or previous values (e.g., revert to revised parameters), maintain the VT/VF detection parameters as adjusted, or set a completely different set of VT/VF detection parameters based on the analysis. The method 300 includes evaluating or determining whether the adjusted VT/VF detection parameters were effective or successful (block 302) during the adjustment time period. In other words, the method 300 may evaluate an effectiveness of the adjusted VT/VF detection parameters (block 302). Success or effectiveness of the adjusted VT/VF detection parameters may be determined in multiple ways.

For example, the method 300 may analyze various physiological parameters (block 302) that were monitored during the adjustment time period (e.g., the time period during which the adjusted VT/VF detection parameters were used). If the various physiological parameters indicate that the patient was stable or uncompromised (e.g., hemodynamically uncompromised) during the adjustment time period, then the method 300 may determine that the adjusted VT/VF detection parameters were effective and/or successful (block 302) and may proceed to maintaining the adjusted VT/VF detection parameters (block 304). In other words, instead returning or reverting the detection parameters to nominal values, the detection parameters may remain as adjusted after the expiration of the adjustment time period (block 218).

In at least one embodiment, evaluating whether the adjusted VT/VF detection parameters were effective and/or successful (block 302) may include analyzing the pressure activity of the patient's heart and/or analyzing the perfusion of the patient monitored during the adjustment time period. If the pressure activity and/or perfusion indicate that the hemodynamic functionality of the patient's heart was not compromised (e.g., the hemodynamic functionality of the patient's heart was adequate) during the adjustment time period, then it may be determined that the adjusted VT/VF detection parameters were successful or effective and the adjusted VT/VF detection parameters may be maintained (block 304). In the alternative, if the pressure activity and/or perfusion indicate that the hemodynamic functionality of the patient's heart was compromised (e.g., the hemodynamic functionality of the patient's heart was inadequate) during the adjustment time period, then it may be determined that the adjusted VT/VF detection parameters were unsuccessful and/or ineffective and the VT/VF detection parameters may be reverted to previous VT/VF detection parameters (e.g., VT/VF detection parameters prior to the last automatic adjustment, nominal VT/VF detection parameters, etc.) (block 202). In at least one embodiment, the pressure activity, perfusion, and/or other various parameters monitored before the VT/VF may be used as baseline information to be compared with the various parameters of the patient monitored during the adjustment time period in making the determination of whether the adjusted VT/VF detection parameters were successful/effective or unsuccessful/ineffective.

Further, for example, the method 300 may receive data indicative of the effectiveness of the adjusted VT/VF detection parameters from an external device via telemetry. If the received data indicates that the patient was stable during the adjustment time period, then the method 300 may determine that the adjusted VT/VF detection parameters were effective and/or successful (block 302) and may proceed to maintaining the adjusted VT/VF detection parameters (block 304).

In at least one embodiment, a patient may provide information indicative of the effectiveness of the adjusted VT/VF detection parameters using an external device to the IMD 16. For instance, if the patient did not experience any dizziness, syncope episodes, and/or any other symptoms indicative of at least one heart condition during the adjustment time period, then the patient may transmit data to the IMD 16 that indicates that the adjusted VT/VF detection parameters were effective and/or successful. Upon receiving the data, it may be determined that the adjusted VT/VF detection parameters were successful and/or effective (block 302) and the adjusted VT/VF detection parameters may be maintained (block 304). In the alternative, the patient may provide information to the IMD 16 that indicates that the adjusted VT/VF detection parameters were ineffective and/or unsuccessful. In this case, the VT/VF detection parameters may be reverted to previous VT/VF detection parameters (e.g., VT/VF detection parameters prior to the last automatic adjustment, nominal VT/VF detection parameters, etc.) (e.g., the method 300 may proceed to providing the VT/VF detection parameters (block 202) of method 200).

If a VT/VF is detected using the maintained, adjusted VT/VF detection parameters (block 306), the method 300 may return to the method 200 to deliver cardiac therapy (block 208) and the remainder of the processes of method 200, such as initiating adjustment of the VT/VF detection parameters (block 214), may be implemented. In essence, the maintained, adjusted VT/VF detection parameters may again be automatically adjusted for at least an adjustment time period. Further, method 200 may again return to method 300 such that the presently-used VT/VF detection parameters may be evaluated and possibly maintained.

The techniques described in this disclosure, including those attributed to the IMD 16, the programmer 24, or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by one or more processors to support one or more aspects of the functionality described in this disclosure.

All patents, patent documents, and references cited herein are incorporated in their entirety as if each were incorporated separately. This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed:

1. An implantable medical device for use in delivering therapy to a patient's heart comprising:
sensing apparatus configured to monitor physiological parameters of a patient, wherein the sensing apparatus comprises at least one electrode to monitor the electrical activity of the patient's heart;
a sensing module coupled to the sensing apparatus and configured to receive the monitored physiological parameters;
a therapy delivery module configured to deliver cardiac therapy to the patient; and
a control module coupled to the sensing module and to the therapy delivery module and configured to:
provide one or more VT/VF detection parameters usable to detect at least one cardiac condition, wherein the at least one cardiac condition comprises at least one of ventricular tachycardia and ventricular fibrillation,
detect the at least one cardiac condition based on the one or more VT/VF detection parameters using the monitored physiological parameters, and
initiate an automatic adjustment of at least one of the one or more VT/VF detection parameters to raise a threshold for detection of the at least one cardiac condition in response to delivery of cardiac therapy to treat the at least one cardiac condition, wherein the automatic adjustment results in one or more adjusted VT/VF detection parameters.

2. The device of claim 1, wherein the automatic adjustment of at least one of the one or more VT/VF detection parameters follows termination of the at least one cardiac condition.

3. The device of claim 1, wherein the one or more VT/VF detection parameters comprise a number of intervals to detect VT/VF, and wherein the automatic adjustment of at least one of the one or more VT/VF detection parameters comprises increasing the number of intervals to detect VT/VF to raise the threshold for detection of the at least one cardiac condition.

4. The device of claim 1, wherein the one or more VT/VF detection parameters comprise a detection interval, and wherein the automatic adjustment of at least one of the one or more VT/VF detection parameters comprises adjusting the detection interval to raise the threshold for detection of the at least one cardiac condition.

5. The device of claim 1, wherein the one or more VT/VF detection parameters comprise an EGM morphology matching score, and wherein the automatic adjustment of at least one of the one or more VT/VF detection parameters comprises adjusting the EGM morphology matching score to raise the threshold for detection of the at least one cardiac condition.

6. The device of claim 1, wherein the control module is further configured to revert to previous one or more VT/VF detection parameters from the one or more adjusted VT/VF detection parameters after expiration of an adjustment time period.

7. The device of claim 1, wherein the control module is further configured to:
evaluate an effectiveness of the one or more adjusted VT/VF detection parameters used during an adjustment time period, wherein the adjustment time period is a selectable time period beginning after the automatic adjustment of the at least one of the one or more VT/VF detection parameters,
revert to previous one or more VT/VF detection parameters from the one or more adjusted VT/VF detection parameters based upon the evaluation of effectiveness of the one or more adjusted VT/VF detection parameters, and
maintain the one or more adjusted VT/VF detection parameters based upon the evaluation of effectiveness of the one or more adjusted VT/VF detection parameters.

8. The device of claim 7, wherein the sensing apparatus further comprises at least one of a pressure sensor to monitor pressure activity of the patient's heart and a perfusion sensor to monitor tissue perfusion of the patient, and wherein the control module is further configured to evaluate the effectiveness of the one or more adjusted VT/VF detection parameters by analyzing at least one of the pressure activity of the patient's heart and the tissue perfusion of the patient monitored during the adjustment time period.

9. The device of claim 1, wherein the control module further comprises a telemetry module configured to communicate with an external device, and wherein the control module is further configured to:
receive data indicative of an effectiveness of the one or more adjusted VT/VF detection parameters from an external device using the telemetry module,
revert to the previous one or more VT/VF detection parameters from the one or more adjusted VT/VF detection parameters based upon the received data indicative of the effectiveness of the one or more adjusted VT/VF detection parameters, and
maintain the one or more adjusted VT/VF detection parameters based upon the received data indicative of the effectiveness of the one or more adjusted VT/VF detection parameters.

10. The device of claim 1, wherein the one or more VT/VF detection parameters comprise a duration of continuous detection to detect VT/VF, and wherein the automatic adjustment of at least one of the one or more VT/VF detection parameters comprises increasing the duration of continuous detection to detect VT/VF to raise the threshold for detection of the at least one cardiac condition.

11. A method for use in delivering therapy to a patient's heart comprising:
monitoring physiological parameters of a patient;
providing one or more VT/VF detection parameters usable to detect at least one cardiac condition, wherein the at least one cardiac condition comprises at least one of ventricular tachycardia and ventricular fibrillation;
detecting the at least one cardiac condition based on the one or more VT/VF detection parameters using the monitored physiological parameters; and
initiating an automatic adjustment of at least one of the one or more VT/VF detection parameters to raise a threshold for detection of the at least one cardiac condition in response to delivery of cardiac therapy to treat the at least one cardiac condition, wherein the automatic adjustment results in one or more adjusted VT/VF detection parameters.

12. The method of claim 11, wherein the automatic adjustment of at least one of the one or more VT/VF detection parameters follows termination of the at least one cardiac condition.

13. The method of claim 11, wherein the one or more VT/VF detection parameters comprise a number of intervals to detect VT/VF, and wherein the automatic adjustment of at least one of the one or more VT/VF detection parameters comprises increasing the number of intervals to detect VT/VF to raise the threshold for detection of the at least one cardiac condition.

14. The method of claim 11, wherein the one or more VT/VF detection parameters comprise a detection interval, and wherein the automatic adjustment of at least one of the one or more VT/VF detection parameters comprises adjusting the detection interval to raise the threshold for detection of the at least one cardiac condition.

15. The method of claim 11, wherein the one or more VT/VF detection parameters comprise an EGM morphology matching score, and wherein the automatic adjustment of at least one of the one or more VT/VF detection parameters comprises adjusting the EGM morphology matching score to raise the threshold for detection of the at least one cardiac condition.

16. The method of claim 11, further comprising reverting to previous one or more VT/VF detection parameters from the one or more adjusted VT/VF detection parameters after expiration of an adjustment time period.

17. The method of claim 11, further comprising:
evaluating an effectiveness of the one or more adjusted VT/VF detection parameters used during an adjustment time period, wherein the adjustment time period is a selectable time period beginning after the automatic adjustment of the at least one of the one or more VT/VF detection parameters; and
reverting to previous one or more VT/VF detection parameters from the one or more adjusted VT/VF detection parameters based upon the evaluation of effectiveness of the one or more adjusted VT/VF detection parameters or maintaining the one or more adjusted VT/VF detection parameters based upon the evaluation of effectiveness of the one or more adjusted VT/VF detection parameters.

18. The method of claim 17, wherein evaluating the effectiveness of the one or more adjusted VT/VF detection parameters comprises monitoring at least one of pressure activity of the patient's heart and tissue perfusion of the patient monitored during the adjustment time period.

19. The method of claim 11, further comprising:
receiving data indicative of an effectiveness of the one or more adjusted VT/VF detection parameters; and
reverting to previous one or more VT/VF detection parameters from the one or more adjusted VT/VF detection parameters based upon the received data indicative of the effectiveness of the one or more adjusted VT/VF detection parameters or maintaining the one or more adjusted VT/VF detection parameters based upon the received data indicative of the effectiveness of the one or more adjusted VT/VF detection parameters.

20. The method of claim 11, further comprising adjusting at least one parameter of cardiac therapy to treat the at least one cardiac condition based on the automatic adjustment of at least one of the one or more VT/VF detection parameters, wherein the at least one parameter of cardiac therapy comprises at least one of an energy level of defibrillation therapy, shock therapy charge time, and an energy level of nerve stimulation.
